(19) United States
(12) Patent Application Publication
(10) Pub. No.: US 2007/0092978 A1
(43) Pub. Date: Apr. 26, 2007

(54) Target ligand detection

(76) Inventors: Ronald Mink, West Linn, OR (US); Toby Gottfried, Orinda, CA (US); John Ennis, Vancouver, WA (US); Paul Smith, Washougal, WA (US); Glen Ford, Montgomery Village, MD (US)

Correspondence Address: KEVIN FARRELL;PIERCE ATWOOD, ONE NEW HAMPSHIRE AVENUE, PORTSMOUTH, NH, 03801, US

(21) Appl. No.: 11/254,975

(22) Filed: Oct. 20, 2005

(51) Int. Cl. G01N 33/543

(52) U.S. Cl. 436/518

(57) ABSTRACT

The present invention provides compositions, devices and methods suitable for the increased sensitivity and selectivity of binding assays thereby reducing false positive results without little or no reduction in the detection of true positives. The present invention is based on the novel discovery that an oxidative agent in the context of the device of the present invention results in decreased false positive reactivity with little or no reduction in true positive reactivity. The devices, compositions and methods of the present invention may be used, for example, to detect pathogens giving rise to endogenous urine antibodies include those organisms known to be causative agents in sexually-transmitted diseases and other diseases. The devices and methods of the present invention are also useful for various diagnostic procedures.

### FIELD OF THE INVENTION

The present invention relates to immunochemistry and biochemical analysis, providing devices and methods suitable for increased sensitivity in detecting target ligands. In particular, the devices and methods of the present invention are suitable for the rapid detection of endogenous urine antibodies, particularly antibodies directed against HIV viral coat proteins. Other pathogens giving rise to endogenous urine antibodies and, therefore, detectable using the present invention include those organisms known to be causative agents in sexually-transmitted diseases as well as other diseases.

### BACKGROUND OF THE INVENTION

Many assays utilizing binding agents specific for target antigens suffer from inadequate sensitivity and selectivity. Although this prior art limitation is not limited to any particular type of assay that utilizes binding agents and target ligands, one type of assay that exemplifies this limitation are lateral flow assays which are used to detect the presence of various substances in body fluids such as urine, oral fluid or blood. These assays typically involve antigen-antibody reactions, synthetic conjugates comprising enzymatic, fluorescent or visually observable tags and specially designed reactor chambers. In most of these assays, there is a receptor (e.g., an antibody), which is specific for the selected antigen, and a means for detecting the presence and/or amount of the antigen-antibody reaction product. Most current tests are designed to make a quantitative determination but, in many circumstances, all that is required is a positive/negative indication. Examples of such qualitative assays include disease detection, blood typing, pregnancy testing and many types of urinalysis. For these tests, visually observable indicia such as the presence of agglutination or a color change are preferred.

The positive/negative assays must be very sensitive because of the often small concentration of the ligand of interest in the test fluid. False positives can be troublesome, particularly with agglutination and other rapid detection methods such as dipstick and color change tests. Because of these problems, sandwich assays and other sensitive detection methods, which use metal sols or other types of colored particles, have been developed. These techniques have not solved all of the problems encountered in these rapid detection methods; however, as they can be costly to manufacture, difficult for non-technical persons to use and still have an unacceptable level of false positive results.

Therefore, a need still exists for detection methods that are both sensitive and selective in detecting, in general, target ligands via binding agents. Also, a need still exists for detection methods that are both sensitive and selective in detecting, in particular, target analytes present in body fluids at small concentrations. A need also exists for such assays that are relatively inexpensive to manufacture, easy to use and also decrease the problems encountered with the generation of false positive results while having little or no reduction in true positive results.

### SUMMARY OF THE INVENTION

The present invention provides devices and methods for the detection of target ligands by labeled binding agents that are more sensitive and more selective than prior art devices and methods. The inventors have found that by operating assays involving target ligands and binding agents in an oxidized environment, much more sensitive and selective assays than are currently available can be carried out. For example, the present invention provides improved devices and methods suitable for the rapid detection of endogenous bodily fluid (e.g., urine) antibodies, particularly, but not limited to, antibodies directed against HIV viral coat proteins. Accordingly, in one embodiment, the present invention provides a lateral flow device for the detection of target antibodies in, for example, urine, the device comprising an antigen that specifically binds the target antibodies and an oxidative agent that is activated upon contact with the sample fluid. In one embodiment, the oxidative agent comprises, for example, one or more reagents selected from the group consisting of hydrogen peroxide ranging from about 0.04-0.4%, urea hydrogen peroxide ranging from about 0.1-0.5% stabilized with about 3% potassium stannate, potassium iodate ranging from about 0.1-0.4%, sucrose (about 1%) with about 0.4-0.8 mg/ml glucose oxidase, potassium superoxide from about 0.4%, Thimerosol ranging at about 0.4-0.6%, Calcium Bromate ranging from about 0.045-0.18% and potassium permanganate ranging from about 0.4%.

It will be apparent to those practiced in the art that both the devices and methods of the present invention are useful in numerous types of assays. These embodiments are part of the present invention and are described in greater detail below in the Detailed Description of the Invention.

The present invention is not limited to the nature of the device used in the context of this invention. In one general embodiment, the present invention contemplates that any binding assay is compatible with the devices and methods of the present invention. For example, the target ligands or binding agents may be proteins, lipids, carbohydrates, glycoproteins, lipoproteins, etc. Any detection device may be used in the present invention including lateral flow devices, petri plates (including multi-well plates [e.g., ouchterlony plates]), chromatography columns, assay tubes (including Eppendorf® tubes), etc. In one embodiment, the device comprises a lateral flow device wherein the sample is applied directly to the sample application area (sample zone) of the device. In another embodiment, the device comprises a sample device used in conjunction with an analyzing (result detection) device. In one aspect of this embodiment, the sample device is used to collect the sample at the sample-collecting zone of the sample device and to apply the sample to the sample-receiving zone of the analyzing device. In another aspect of this embodiment, the analyzing device is a lateral flow device. In yet other embodiments, the invention contemplates using the methods of the present invention in any device in which an assay is performed where a binding agent is bound to a target ligand.

The devices and methods of the present invention are also cost-effective, as they maximize the use of low cost reagents, such as non-specific antibody binding proteins like protein A, protein G or lectins, in the immunoassays of the invention.

Some assay devices of the present invention comprise a series of distinct zones defined by the reagents and/or reactions that take place within the respective zones during the operation of the device. The zones may be part of a single continuous matrix, or incorporated into two or more discrete pads that are brought into fluid communication in the claimed device. Details are discussed below in the Detailed Description of the Invention.

### DETAILED DESCRIPTION OF THE INVENTION

In one aspect, the invention relates to compositions and methods to increase the sensitivity of assays by operating the assays in an oxidative environment. Although not limited by any theory, it is believed that the oxidative environment increases binding agent/target ligand interactions thereby allowing for the detection of smaller amounts of target ligand than would be possible when the assay is not operated in an oxidative environment. For example, in one embodiment, the present invention contemplates a method for detecting the presence or absence of a target ligand in a sample, the method comprising a labeled binding agent characterized by the ability to bind a target ligand; contacting the labeled binding agent with a sample suspected of containing the target ligand under conditions suitable for binding the target ligand to the labeled binding agent, wherein contact of the labeled binding agent with the sample occurs in the presence of an oxidizing agent and, then, assessing the binding of the target ligand to the labeled binding agent, thereby detecting the presence or absence of the target ligand in the sample.

In other aspects, the sample or target ligand can be any sample or be found in any sample including, but not limited to, biological (e.g., proteins, lipids, carbohydrates), chemical, synthetic, etc. In a preferred embodiment, the target ligand or sample is biological. Examples are tissues, biopsies and bodily fluids. Examples of bodily fluids with which devices, compositions and methods of the present invention are compatible are urine, blood (including plasma), spinal fluid, oral fluid, semen, lymph fluid, etc.

The target ligand, as mentioned above, may be a protein, lipid, glycoprotein, lipoprotein, carbohydrate, nucleic acid, etc. If the target ligand is a protein, any protein is contemplated as an appropriate target ligand for the compositions and methods of the present invention. In preferred embodiments, the target ligand is an antibody, antigen or a hormone. In one embodiment, the target ligand is a hormone indicative of pregnancy. In a more preferred embodiment, the target ligand is an antibody to an HIV antigen.

In other embodiments, the target ligand is a non-protein. Examples of non-protein target ligands are lipids such as those found in cellular membranes (e.g., fatty acids, glycerophospholipids, sphingolipids, steroids, triglycerides and cholesterol) or other biological systems. In a preferred embodiment, the present invention contemplates that compositions and methods of the present invention are useful in the detection of steroids, hormones and cholesterol. Binding agents for non-protein target ligands are known in the art and may include antibodies, enzymes (and other natural or synthetic interactive molecules), etc. In other aspects, the non-protein target ligand is a carbohydrate (e.g., glucose, disaccharides, polysaccharides, etc.). In one embodiment, the present invention contemplates that the compositions and methods of the present invention an useful in the diagnosis and monitoring of diabetes.

In some aspects of the invention, the assay takes place in solution (e.g., for the binding of soluble target ligands). In some aspects of the present invention, the assay takes place on a solid support that is suitable for the binding of the target ligand to the labeled binding agent. In some instances, the target ligand is bound to the solid support prior to detection by the labeled binding agent. In other instances, the labeled binding agent is bound to the solid support prior to binding the target ligand. In other aspects, the labeled binding agent or target ligand is movably bound to the support such that it may migrate along the solid support when, for example, a fluid passes over or through the solid support. As described below, the solid support may have porosity in order to allow for the movement of target ligands and/or labeled binding agents. In one embodiment, the solid support that allows for the movability of the target ligand and/or labeled binding agent is a chromatographic strip. Other types of solid supports are contemplated for the present invention and are described in detail below and include, but are not limited to, filter paper, nitrocellulose, and various other chromatography media (including beads, papers and synthetic materials).

In other embodiments, the present invention provides devices and methods suitable for the rapid detection of endogenous urine antibodies, particularly antibodies directed against HIV viral coat proteins. Other pathogens giving rise to endogenous urine antibodies and, therefore, detectable using the present invention include, for example, those organisms known to be causative agents in sexually-transmitted and infectious diseases. Exemplary causative agents and disease states detectable using the present invention include Chlamydia, herpes virus, gonorrhea, syphilis, Helicobacter pylori, hepatitis A, C, and H viruses, EBV, CMV, HSV, malaria, influenza, West Nile virus, Rubella, Dengue fever, Lyme disease, Chagas, tuberculosis, toxoplasmosis, Ebola and the like. The devices utilize agents capable of initiating and/or maintaining an oxidative environment in the lateral flow device. The Inventors have discovered that by conducting the assay in an oxidative environment, the sensitivity and specificity of the present invention is dramatically increased over prior art devices and methods since the visual signal presented by the label of the invention is substantially enhanced over the signal presented in a non-oxidative environment. While the assay discussed in detail below is conducted in a lateral flow format, it will be recognized by one skilled in the art that the establishment of an oxidative environment for such assays will improve detection irrespective of the particular assay format selected.

To further ease in understanding the terminology of the present invention, the following definitions are provided:

I. Definitions

An “analysis zone” is a region of a flow path that includes an immobilized antigen that specifically binds a target antibody endogenous to the urine sample being tested. Specific binding of the target antibody by the antigen retains the target antibody, and any molecule associated with it, in the analysis zone.

A “sample zone” is a region of a flow path of an analyzing device wherein the sample to be analyzed is deposited. In the context of the present invention, the sample zone may include an oxidizing agent. A “sample collecting zone” is a region of a sample device where a sample may be collected for the purpose of application to the sample zone on the analyzing device.

A “conjugation zone” is a region of a flow path that comprises a labeled agent suitable for binding to antibodies contained in the sample. Examples of such agents are described below.

A “control zone” is a region of a flow path that includes an immobilized antigen that specifically binds a target antibody endogenous to the urine sample being tested. Specific binding of the target antibody by the antigen retains the target antibody, and any molecule associated with it, in the control zone. The control zone differs from the analysis zone in that the analysis zone is specific for the antigen that is indicative of the condition being tested whereas the control zone is not specific for any particular antibody and serves only to indicate that the sample contains antibody and the device is working properly.

A “capture agent” is any molecule that specifically binds a target antibody. Capture reagents of the present invention are preferably immobilized to the matrix in a defined pattern, typically a line perpendicular to the flow path. Preferred capture reagents are anti-target antibody antibodies, protein A and protein G.

“Matrix” refers to an insoluble material capable of supporting fluid flow. Matrix materials may be from natural and/or synthetic sources, bibulous or non-bibulous, fibrous or particulate. Matrices of the invention may be formed as continuous strips of the same material or mixtures of different materials that are distributed consistently along a common strip, or inconsistently such as to form zones having different physical or chemical characteristics in different regions of the strip. Alternatively, a series of discrete pads can be formed from the same or different matrix materials, with reagents for the assay being added to each pad. The pads may then be placed in fluid communication with each other to form a continuous flow path. Materials used to construct matrices of the invention may be inert or may react with one or more reagents of the invention, provided that the materials remain insoluble during the practice of the invention as described herein.

“Downstream” refers to the directional flow path of a liquid, through a matrix, away from the point of liquid application.

“Upstream” refers to the directional flow path of a liquid, through a matrix, toward the point of liquid application.

“Flow path” refers to the route taken by a urine sample as it passes through a matrix. The flow path is preferably a single route, but may include several routes where each route may support liquid flow simultaneously, sequentially or independently relative to other routes.

II. Introduction

As summarized above, the devices of the present invention are designed to detect the presence of target ligands from, for example, a patient sample. The invention is not limited to the nature of the target ligand. For example, the target ligand may be a protein (e.g., an antibody or antigen), a lipid (e.g., cholesterol, steroids), a carbohydrate (e.g., glucose) or a hormone (e.g., estrogen). In one example, the devices of the present invention are designed to detect the presence of target antibodies endogenous to, for example, a patient's urine sample. Target antibodies preferably recognized by devices of the invention are those antibodies that specifically bind HIV proteins. In some embodiments, the HIV is HIV-1 while in other embodiments the HIV is HIV-2. In still other embodiments, more than one antigen is utilized with the multiple antigens being from HIV-1 and/or HIV-2. In certain aspects of the embodiment, the protein antigen is recombinantly produced. In preferred embodiments the HIV protein is an envelope protein. In another preferred embodiment, the protein is a non-native peptide (i.e., synthetic) of gp120 or gp41 from HIV-1 or gp36 from HIV-2. Still other embodiments include multiple HIV proteins including any and all combinations of the peptides presented above. In some embodiments, the antigen is biotinylated and linked to the matrix of the device through a strepavidin-biotin or avidin-biotin linker.

In another embodiment, the lateral flow devices of the present invention comprise, for example, in addition to a sample zone: (a) a conjugation zone comprising a Protein A/colloidal gold conjugate and (b) a control zone comprising a capture agent, wherein the capture agent has an affinity for a human urine antibody bound to the Protein A/colloidal gold conjugate that is greater than the affinity of Protein A for the human urine antibody bound to the Protein A/colloidal gold conjugate. This embodiment may have a capture agent that is an anti-human IgG antibody and preferably a goat anti-human IgG antibody. Another embodiment of the present invention is the addition of a sample zone comprising an antigen that specifically binds the target antibodies. Several aspects of the present embodiment also have a sample zone that further comprises serum (for example, bovine, porcine, avian serum [preferably chicken]or other serum). Although the present invention is not limited by theory, it is believed that the serum functions as a blocking agent.

In one general embodiment, the present invention provides an immunochemical sampling device to be used in conjunction with the lateral flow device of the present invention, said sampling device comprising an elongated support (core stick) surrounded at one end of its proximal ends by a porous layer, optionally with an impermeable protecting layer wherein said porous layer comprises a labeled specific binding reagent which is activated by the liquid sample and mobilized in a controlled manner when the sample device has been contacted with a analyzer device comprising a porous carrier.

In another aspect, the porous layer comprises a labeled specific binding reagent and has been treated with a blocking solution preventing reactive groups of the porous material to react with the liquid to be tested and, subsequently, brought in contact with a porous carrier of the analyzer comprising at least one specific binding reagent.

The sampling device may be contacted with the liquid to be tested and subsequently brought in contact with a porous carrier of the analyzer device comprising at least one specific binding reagent immobilized as a dot or zone.

In another aspect, the elongated support (core stick) of the sampling device is an elongated stick made out of wood or plastic. For example, polypropylene (PP) or polyvinylchloride (PVC) may be used. The sampling device is surrounded at its proximal end by a porous layer and an impermeable layer comprising one or more and preferably 1 to about 5 layers of tape (or similar) for adjusting the flow of the labeled binding reagent from the sampling device to the analyzer device. The porous layer of the sampling device also comprises one or more layers, preferably 1 to about 5 layers of a porous material. The porous material may be selected from a group of material consisting of, for example, paper, glass fiber, nylon, polyester or cellulose and derivatives thereof.

In another aspect, the porous layers of the sampling device comprise at least one specific labeled binding reagent. The labeled binding agent or agents are impregnated in either a part or the whole of the porous material of the sampling device.

In another aspect, the porous carrier of the analyzer device comprises at least one specific binding reagent directly or indirectly immobilized as a dot or zone (test line). Moreover, one or more dots or zones on the porous carrier may act as control zones. The sampling device can be used in connection with an analyzed device where the porous carrier comprises one porous passage, which may be penetrated by a sample solution, containing detection zones(s) but also with an analyzer device where the porous carrier comprises two or more channels optionally made by a suitable method comprising at lest one specific binding reagent per channel, immobilized as a dot or zone. The porous material of the analyzer device is selected from a group of materials consisting of, for example, nitrocellulose, paper, glass fiber, nylon, polyester, polysulphonate or cellulose and derivatives thereof.

The analyzer device may be of various forms. For example, the device may comprise one or more channels to enable the testing of several analytes simultaneously. Markers specific for different analytes can be grouped together, for example, to form different diagnostic tests on the same analyzer device. The multiple channel analyzer device comprises a porous carrier processed by a water-repellency treatment or otherwise in order to cause a network of channels where the tested sample can migrate. Different specific binding reagents may be bound in each channel.

The specific binding reagents of the sampling device and/or the analyzer device include, but are not limited to, antibodies, antibody fragments, recombinant antibodies, recombinant antibody fragments, antigens, lectins, receptors and/or ligands. The type of labels useful in the sampling device and/or the analyzer device include colored latex, gold, metal, dye, fluorogenic substances, superpara-magnetic particles coated with the specific binders. Chromogenic substances, particularly fluorochromogens and enzymatic labels may be used as markers as well.

The blocking material for making the porous material inert is, for example, a mixture comprising natural or synthetic polymers such as albumin (BSA, bovine serum albumin) and casein or PEG (polyethylene glycol), PVA (polyvinyl alcohol) and PVP (polyvinyl pyrrolidone), nonionic detergents such as HEXA (hexane sulphonic acid) and TRITON-X-100, SDS BRIJ and preservation agents such as sugar (e.g., glucose, sucrose and trehalose or derivatives thereof).

In one embodiment, the sampling device is dried to a moisture content of about 8% or less and packed hermetically and separately or in combination with said analyzer device.

The detection of an analyte in a liquid sample is achieved by bringing the sample device into contact with the liquid sample and, then, with the analyzer device. The liquid sample alongside with the labeled specific binding reagent is allowed to migrate from the diagnostic sampling device to the porous carrier of the analyzing device from which the positive or negative results are directly readable. The result can be read directly visually (by eye) or by appropriate equipment capable of recording the results.

The invention further provides a detection system comprising, for example, an immunochemical sampling device where the device is contacted or left in contact with the sample zone of the analyzer device. The liquid sample and the labeled specific binding reagent, or the reaction product (complex) formed thereof, are allowed to migrate or flow from the sampling device to the porous carrier of the analyzer device. The analyzer device may also comprise a specific binding reagent on the porous carrier. Normally, the liquid from the sampling device moves throughout the porous carrier of the analyzer device by diffusion and/or capillary action.

In one embodiment, the liquid sample can be urine, blood, serum, plasma, semen, oral fluid or a sample buffer solution. In the case of viscous sample, a dilution step with an appropriate buffer is contemplated. In a particularly preferred embodiment, the liquid sample is urine.

Besides the increased accuracy of the test results, other advantages of the combination sampling device/analyzer device system of the invention are the small format of the sampling device and the analyzer device which leads to material savings, less waste products and decreased freight coasts and, thus, environmentally friendlier products. The device does not require refrigeration although it may be. Further, as the test system is easy to use it enables home use. Since analyzer device is not in direct contact with the liquid sample, overflow is avoided and an increased reliability of the test is obtained. Moreover, the sampling device and analyzer device of the invention are easy to store since the devices are dried and that they are possible to store hermetically. Also, the immunochemical sampling device described above enables a controlled application of the sample and/or labeled specific binding reagent on to the analyzer device.

In another embodiment, the analytes to be detected by the devices and methods of the present invention are, for example, disease specific antibodies including IgG, IgM and IgA, antibodies against Helicobacter pylori, hepatitis A, HIV_{1,2}, respiratory disorders, etc. Antigens excreted into urine include luteinizing hormone (LH), follicle stimulating hormone (FSH) and human chorionic gonadotropin (hCG) or, for example, antigens of or antibodies against bacteria, virus, fungi and parasites or components and products thereof. The devices and methods of the present invention may be used for a wide variety of different tests including pregnancy, menopause, fertility, thyroid stimulating hormone, toxoplasmosis, cancer antigens, respiratory disorders, allergies, myocardial infarcts, drug tests, sexually transmitted diseases, etc.

All embodiments of the present invention may be provided in kit form and packaged with one or more ancillary articles as described herein or known in the art.

Another embodiment of the present invention is a method for detecting antibodies in a urine sample from a patient, the method comprising contacting the sample with a sample zone of an analyzer device, wherein the sample zone comprises an oxidative agent. Various aspects of this method embodiment utilize the same variety of buffers and antigens as the previous embodiments.

Yet another embodiment of the invention is a method for detecting antibodies in a urine sample from a patient, the method comprising contacting the sample with a sample zone on an analyzer device wherein the lateral flow device further comprises a conjugation zone that comprises a Protein A/colloidal gold conjugate, an analysis zone and a control zone that comprises a capture agent, wherein the capture agent has an affinity for a human urine antibody bound to the Protein A/colloidal gold conjugate that is greater than the affinity of Protein A for the human urine antibody bound to the Protein A/colloidal gold conjugate. Again, this embodiment shares the same variety of aspects as previous embodiments with regard to antibodies, antigens and buffers.

The devices of the invention comprise a series of zones, each distinguished by the chemical reagents, reactions and/or interactions occurring in the respective zone. At a minimum, each device comprises at least three zones; the sample zone; the conjugation zone; and the analysis zone. Devices of the invention may also include a control zone to indicate the presence of antibody in the sample and that the device has performed properly in operation and is providing a valid result. Devices also may optionally include a waste region beyond the last zone in the flow path, where excess sample may accumulate. The waste region provides the ability to add additional sample volume to the device when necessary. Typically the waste region includes an absorbent material, which may be the same material as the matrix forming any or all of the zones of the device.

The devices of the present invention may also comprise a sampling device. The sampling device is used to collect the specimen sample and apply it to the analyzing device. The sampling device contains oxidizing agents and buffers used to, e.g., modify the biological specimen. The use of the sampling device allows for, e.g., a more controlled application of the sample to the analyzing device. The use of a sampling device may be preferred for collecting samples from, e.g., children, the elderly, the infirmed or, e.g., when the precise application of the sample to the analyzing device may be critical to ensure accurate test results. The analyzing device comprises the sample, conjugation, analysis zones and, optionally, the control zone and waste region, as described above.

The following sections provide a detailed description of zones found in devices of the present invention, and how these zones are arranged along a flow path to produce a valid diagnostic result. By way of this description, methodology for operating the devices will also be apparent.

III. Analyzing Device Construction

Generally, the devices of the invention are designed to allow sequential flow of a urine sample along a flow path comprising each zone of the device. Thus a urine sample applied to the sample zone will sequentially encounter the reagents in each subsequent zone thereby allowing a predetermined series of reactions to occur between sample constituents and the reagents present in each zone. Liquid flow through the device is controlled by a matrix material that performs a number of functions, as described below. The matrix material may optionally be placed in a housing, also discussed below. After discussion of the matrix and housing, zones that may be incorporated in each device will be described individually in the order in which they are encountered by a urine sample as it traverses a flow path of the device.

A. Matrix

Matrices suitable for use in the analyzing devices of the present invention are insoluble materials capable of supporting fluid flow. Matrix materials may be from natural and/or synthetic sources, are porous, bibulous or non-bibulous, fibrous or particulate. They may be formed as continuous strips of the same material, mixtures of different materials that are distributed consistently along a common strip, or inconsistently distributed mixtures that form regions having different physical or chemical characteristics in different areas of the strip. Alternatively, the matrix may be formed from two or more pads of matrix material. The pads are then orientated in fluid communication with each other to form a flow path of the device. Constructing the flow path from a series of pads is particularly useful when the device requires a plurality of zones, each comprising a different set of reagents, or prepared using mutually exclusive methods, as is the case for several embodiments of the present invention. Materials used to construct matrices of the invention may be inert or may react with one or more reagents of the invention, provided that the materials forming the matrix remain insoluble during the practice of the invention as described herein.

Suitable matrix materials are generally hydrophilic, or are capable of being rendered hydrophilic, and include inorganic powders, such as silica and alumina; glass fiber filter paper; natural polymeric materials particularly cellulose-based materials such as filter paper, chromatographic paper, and the like are particularly preferred; synthetic or modified naturally occurring polymers such as nitrocellulose, cellulose acetate, poly(vinyl chloride), polyacrylamide, crosslinked dextran, agarose, etc.; may either be used alone or in conjunction with other materials. The matrix material may also contain functional groups, or be capable of being functionalized to permit covalent bonding of reagents or antigens of the invention.

The matrix material preferably defines the flow path that will be followed by the sample during operation of the device, therefore reagents for use in the assay of the device are typically added directly to the matrix material as a powder or solution, as described below.

1. Oxidative Reagents

An embodiment of the present invention is that the sample flow takes place (in the analyzing device and, optionally, in the sample device) in an oxidative environment. In this regard, agents are added to the analyzing device and, optionally, the sample device that create an oxidative environment for the sample agent to be detected by the particular analyzing device. Agents useful for this purpose in the present invention include, but are not limited to one or more reagents selected from a group consisting of hydrogen peroxide, urea hydrogen peroxide stabilized with about 3% potassium stannate, potassium iodate, sucrose (at about, e.g., 1%) with about 0.8 mg/ml glucose oxidase, Thimerosol, potassium superoxide, potassium perchlorate and potassium permanganate.

In one embodiment, the oxidative agent or agents are added to the analyzing device and, optionally, the sample device after construction of the device.

The oxidative agent or agents are then dried along with the device to approximately 8% or less moisture content. The oxidative agent or agents are then solublized upon contact with the sample.

In another embodiment, the oxidative agent or agents used in the invention are preferably applied to the matrix of a device of the invention prior to operation of the device. The oxidative agent or agents may be applied in any manner that allows the oxidative agent or agents to form a solution having the effect when the matrix is contacted with a sample. For example, the oxidative agent or agents may be applied to the matrix as a dry powder, or preferably applied as a solution, which is subsequently dried or lyophilized in the matrix.

2. Blocking agents

Although inherently bibulous materials may be used as matrix materials in the present invention, fluid flow through the devices of the present invention is preferably nonbibulous in nature.

Bibulous materials may be converted to materials that exhibit nonbibulous flow characteristics by the application of blocking agents. These agents may be detergents, sugars or proteins that can obscure the interactive forces giving rise to the bibulous characteristics. Exemplary protein blocking agents include bovine serum albumin, either per se or in methylated or succinylated form, whole animal sera, such as horse serum or fetal calf serum, and other blood proteins. A preferred blocking agent is avian serum such as goose or turkey serum, most preferably chicken serum. Other examples of protein blocking agents include casein and nonfat dry milk. Detergent-based blocking agents are selected from nonionic, cationic, anionic and amphoteric forms, with the selection based on the nature of the matrix that is being blocked. Tween 20 is a particularly useful detergent for blocking membranes. Exemplary sugars useful as blocking agents include sucrose and fructose.

Application of the blocking reagent to a bibulous matrix may be carried out by treating the matrix with a solution of the blocking agent in an effective concentration to dispose of unwanted reactivities at the surface. In general, this treatment is conducted with a blocking solution, such as a protein solution of 1-20 mg/ml protein at approximately room temperature for between several minutes and several hours. The resulting coated material is then permanently adsorbed to the surface by air-drying, lyophilization, or other drying methods.

The use of a matrix that is inherently bibulous, but convertible to a nonbibulous flow characteristic, is particularly useful for immobilizing antigens and capture reagents. For example, a capture reagent may be applied to the matrix before the application of blocking agents and can be immobilized in situ. Once the capture reagent has been immobilized to the matrix, the blocking agent may then be applied.

B. Housing

Matrices of the analyzing device of the present invention may be disposed within a housing that is both protective and functional. In one preferred embodiment the housing is adapted to have at least one port for receiving a urine sample and guiding fluid flow of the sample to contact the sample zone. The housing also may have windows allowing access to selected portions of the flow path, preferably the analysis zone and/or the control zone. Embodiments having a housing of this type are termed “cassette devices.”

Alternatively, the matrix may be provided unsupported, or supported by a backing formed from a durable material that is preferably impermeable and maintains the physical integrity of the matrix. Embodiments having this type of construction are termed “dip sticks.”

A third device embodiment includes a protective housing analogous to cassette devices, but with a sample zone that extends outside the housing forming a wick that can be dipped into a urine sample. Other variants on these themes are also contemplated and will be readily identified and appreciated by those of skill in the art.

Housings may be constructed of any suitable material known to those of skill in the art. It will be readily appreciated that housing components in fluid contact with the flow path should not impede fluid flow along the flow path and therefore cannot be too hydrophobic. Conversely, the housing material in contact with the flow path cannot be too hydrophilic or the sample may partition to and only traverse the flow path along the walls of the housing.

C. Device zones

Devices of the present invention have at least three zones that include reagents that may interact with antibodies endogenous to a urine sample applied to the device. The sample zone initially receives the urine sample. Application of urine, for example, to the sample zone may be achieved by in stream application, or prior collection of urine followed contact of the sample to the sample zone by dipping, pipetting or pouring the urine sample or the via the application of the sample from a sample device. The urine sample is preferably applied undiluted, and immediately after collection from the patient. If necessary, urine samples to be analyzed using the invention may be stored for a limited period (e.g., up to a week) at room temperature, or for a more prolonged period refrigerated or frozen. Preferably, the sample zone contains an agent or agents sufficient to establish an oxidative environment during operation of the device. The oxidative agent or agents should be readily solubilized by the sample in amounts sufficient to provide the desired oxidative characteristics throughout the operation of the device, as described above.

Sample applied to the sample zone migrates first to the conjugation zone where antibodies endogenous to the urine sample interact with a labeling reagent that is coupled to a label, as described below. This interaction forms a labeled antibody conjugate.

The labeled antibody conjugate then migrates into the analysis zone, where an antigen that specifically binds the target antibody is immobilized to the matrix. If an antibody in the labeled antibody conjugate is a target antibody, the immobilized antigen specifically binds the target antibody, immobilizing the labeled antibody conjugate to the matrix. In this manner label accumulates in the analysis zone, where it can be detected, indicating the presence of the target antibody in the urine sample. If the labeled antibody conjugate does not include the target antibody, it continues along the flow path and label does not accumulate in the analysis zone.

Devices of the invention may optionally include a control zone. Within the control zone is a capture reagent immobilized to the matrix. The capture reagent is deposited to form a control line within the control zone, and binds the labeled antibody conjugate regardless of the nature of the antibody associated with it. This allows the labeled antibody conjugate to accumulate along the control line, accumulating label in the control zone, which indicates that the device is working properly. When present, the control zone is downstream from the conjugation zone, preferably downstream from the analysis zone.

Devices may also optionally include a waste region downstream from all of the zones noted above. The waste region may simply be an extension of the matrix material discussed above, but is preferably constructed from an absorbent material that helps maximize the amount of urine sample that can be applied to the device.

To better describe the invention, each of the zones mentioned in this section is discussed more fully below.

1. Sample Zone

The sample zone receives the sample (e.g., urine) from the operator of the invention. The sample zone is typically constructed of a material that exhibits low target antibody retention. Accordingly, blocking agents of the invention applied to the sample zone in amounts sufficient to prevent target antibody interaction with the matrix material during operation of the invention. A particularly advantageous blocking agent for use in the sample zone is avian sera, more preferably chicken sera. In a preferable embodiment, the sample zone is prepared from a glass fiber pad that is impregnated with a solution containing polyvinylpyrollidone, bovine serum albumin, avian sera, borate and/or carbonate buffers (−0.5M), and triton X-100 or tween-20 detergent. The pad is squeezed to remove excess buffer and the pad is dried overnight at 30° C. An advantage of this approach is increased wetability and wicking action of the sample zone. In some embodiments the sample zone may also function as a mechanical filter, entrapping any undesirable particulates.

Additionally, and at a minimum, the sample zone comprises one or more of the oxidative agents discussed above. In another embodiment, any or all of the other zones (conjugation, analysis and control) may comprise the one or more of the oxidative agents discussed above. Indeed, the entire matrix may contain one or more of the oxidative agents discussed above. As the sample flows through the sample zone, the oxidative agent or agents are solublized and join the fluid flow through the device.

2. Conjugation Zone

The conjugation zone is downstream from the sample zone and contains a label moiety comprising a labeling agent coupled, directly or indirectly, with a label. Methods for coupling labeling agents and labels, as described herein, are well known to those of skill in the art.

The label moiety is deposited in the matrix of the conjugation zone in a manner that allows it to be readily mobilizable in the fluid flow upon contact with a liquid sample, such as a urine sample. To accomplish this, the matrix of the conjugation zone is formed from, for example, a spun-bonded polyester and blocked by dipping it in a buffer containing, for example, polyvinlypyrollidone, chicken serum, bovine serum albumin, carbonate and/or borate buffers. The conjugation zone is then dried at 50° C. and forced air for 50 minutes. The label moiety is striped onto the pad using, for example, either a contact tip or an aerosol tip. Urea Hydrogen Peroxide or other oxidizing agents is added to the label moiety before stripping. Prior to striping, the conjugate is preferably stabilized. For example, the label moiety may be placed in a simple or complex sugar solution, e.g., sucrose at 20% w:v and trehalose at 5% w:v. dextrin at 10%.

As the sample flows through the conjugation zone, the label moiety is solublized and joins the fluid flow through the device. Both suitable labeling agents and labels are discussed further below.

a) Labeling Agents

It is preferable for labeling agents of the invention to specifically bind antibody endogenous to the urine sample. Suitable labeling agents capable of binding any antibody endogenous to the urine sample include bacterial proteins, such as protein G and protein A, and antibodies that recognize particular antibody types. For example, goat anti-human IgG may be used to bind any IgG antibody endogenous to, for example, a urine sample from a human patient.

Regardless of the labeling reagent used, a labeled antibody conjugate will always arise from the conjugation zone, even in the absence of target antibody in the urine sample. If the labeling agent is in the absence of target antibody, then the labeled antibody conjugate will include antibodies generally endogenous to the urine sample, as urine samples are known to include enough endogenous antibody to form a detectable labeled antibody conjugate.

b) Labels

Suitable labels for use in the present invention may or may not be visible, but can be detected if accumulated in the analysis zone. Labels suitable for use in the present invention include, but are not limited to, particulate moieties and enzymes. Visible labels may be dyes or dyed polymers that are visible when present in sufficient quantity. Preferable labels are particles such as dyed latex beads, liposomes, or metallic, organic, inorganic or dye solutions, fluorescent particles, dyed or colored cells or organisms, red blood cells and the like. The metal sol particles, dyed or fluorescent-labeled microparticles should be visible to the naked eye or able to be read with an appropriate instrument (spectrophotometer, fluorescent reader, etc.). Alternatively, radioactive isotopes may also be used.

A preferred label of the present invention are colloidal gold particles that are preferably larger than 10 nm, more preferably in the range of about 20 to 100 nm, and most preferably in the range of 20 to 40 nm. The gold sol particles used in accordance with the present invention may be prepared by methodologies that are well known; e.g., G. Frens, Nature, 241, 20-22 (1973). In addition to gold metal sol, particles may be made of platinum, gold, silver, selenium, or copper or any number of metal compounds which exhibit characteristic colors. Coupling metal, metal compounds and polymer nuclei coated with metals or metal compounds is known in the art and described in U.S. Pat. No. 4,313,734. Other methods well known in the art may be used to attach the analyte to gold particles. The methods include but are not limited to covalent coupling and hydrophobic bonding.

3. Analysis Zone

The analysis zone lies downstream in the flow path from conjugation zone. The analysis zone contains an immobilized antigen that specifically binds the target antibody and in so doing immobilizes the labeled antibody conjugate to the matrix. The immobilized antigen may be any antigen that specifically binds the target antibody, but is preferably an HIV protein, preferably an HIV envelope protein, more preferably one or more peptides from gp120 or gp41 of HIV-1 or gp36 of HIV-2.

Antigen suitable for use in the invention may be obtained from any source including native, chemical synthesis or recombinant production, using methods well known to those of skill in the art. For example, the peptide be chemically synthesized using solid-phase peptide synthesis techniques, or recombinantly produced by operably linking a nucleic acid encoding the desired peptide into an expression vector, and expressing the nucleic acid in a suitable host. Once isolated, the peptide may be biotinylated using known techniques.

Suitable antigens may be immobilized to the matrix using any method known to those of skill in the art that does not destroy specific binding of the antigen to the target antibody. Preferably, the antigen is immobilized to the matrix using a biotin/strepavidin linker, most preferably, the antigen is coupled to biotin and complexed with strepavidin prior to coupling strepavidin to the matrix. Coupling strepavidin of the complex to the matrix is typically done prior to blocking, e.g., for bibulous matrices, using techniques well known to those of skill in the art. Preferably coupling is achieved in a solution containing at least a 2:1 ratio of strepavidin binding site equivalents to each biotin moiety, although other ratios such as 0.5:1, 1:1, 3:1, 4:1 and 5:1, among others and all intermediate (fractional) ratios, are also contemplated as being part of the present invention. For bibulous matrices, the final complex may simply be applied to the matrix material and dried followed by blocking with a suitable blocking agent.

Immobilization of the antigen to the matrix is preferably performed in a manner that serves to concentrate labeled antibody conjugate that specifically binds to the immobilized antigen. By concentrating labeled antibody conjugate, the signal produced by the label is strengthened, improving sensitivity and minimizing the potential of obtaining an erroneous result.

Typically label signals may be observed between 15 and 60 minutes, more preferably between 15 and 45 minutes, most preferably between 15 and 30 minutes after the urine sample is applied to the sample zone. Signals produced by colored labels, as described above, can generally be detected directly from the device without further processing. Fluorescent labels may require a fluorometer to detect. Signals produced by metal sol labels may be enhanced using silver salt solutions in methods well known to those of skill in the art. Similarly, when enzymes are used, the label must be contacted with a substrate of the enzyme label that produces a detectable product. Thus, these enhanced methods deviate from the routine, single-step assay performed with colored particulate labels and sols as the matrix must be contacted with a developing solution (a silver salt or substrate solution) before the label is detected.

4. Control Zone

Devices of the present invention optionally include a control zone. When present, the control zone is down stream in the flow path from the conjugation zone, preferably downstream from the analysis zone.

The control zone contains a capture reagent that specifically binds antibodies endogenous to the sample (e.g., urine) and is preferably immobilized within the control zone to form a control line that concentrates any labeled antibody conjugate bound by the capture reagent. The capture reagent may be a protein having affinity for a class of antibodies, such as protein A or G, but these antibody binding molecules can only be used as the capture reagent of the invention when they are not being used as the inventions labeling agent. Preferred capture reagents have an affinity for endogenous sample antibody that is greater than that of protein A under the operating conditions of the invention. Preferred capture reagents include anti-IgG antibodies from a species other than the one contributing the sample, as described above.

Capture reagents suitable for use in the present invention are immobilized to the matrix using known techniques, including those described above for the immobilized antigen. The capture reagent is preferably immobilized to the matrix using a biotin/strepavidin linker. Most preferably, the capture reagent is coupled to biotin and complexed with strepavidin prior to coupling strepavidin to the matrix, as described above. For bibulous matrices, the matrix material must again be blocked using, for example, a solution containing 0.01M potassium phosphate solution, with 0.25% BSA and 0.025% tween-20. The membrane is then dried overnight at 50° C.

When operating correctly, capture reagent will continue to bind all labeled antibody conjugate until the unbound labeled antibody conjugate is depleted, or the capture reagent is saturated. As even urine samples from healthy mammals contain endogenous IgG, and the molar amount of labeling agent coupled to label preferably exceeds the molar amount of immobilized antigen, labeled antibody conjugate should always be available to bind to capture reagent, producing a signal at the control line. Therefore, failure to detect a signal at the control line is usually indicative of a faulty device or poor operation of the device but may also indicate the absence of IgG in the sample.

IV. Sample Device Construction

The sampling device of the present invention comprises an elongated support (core stick). The elongated support (core stick) of the sampling device is an elongated stick made out of wood or plastic (for example, polypropylene (PP) or polyvinylchloride (PVC)). The sampling device is surrounded at its proximal end by a porous layer and an impermeable layer comprising one or more and preferably 1-5 layers of tape (or similar) for adjusting the flow of the labeled binding reagent from the sampling device to the analyzer device. The porous layer of the sampling device also comprises one or more layers, preferably 1-5 layers of a porous material. The porous material may be selected from a group of material consisting of, for example, paper, glass fiber, nylon, polyester or cellulose and derivatives thereof.

A porous layer is applied around one end of the stick. The porous layer comprises a labeled specific binding reagent that has been treated with a blocking solution (e.g., BSA, casein, serum, etc.) thereby preventing reactive groups of the porous material to react with the liquid to be tested. The sampling device may be contacted with the liquid to be tested and subsequently brought in contact with a porous carrier of the analyzer device comprising at least one specific immobilized binding reagent.

In another embodiment, the porous layers of the sampling device comprise at least one specific labeled binding reagent. The labeled binding agent or agents are impregnated in either part or the whole of the porous material of the sampling device.

V. Kits

The present invention also provides kits that include the one or more devices described above. Each kit may optionally include a package insert providing instruction on the use of the enclosed device(s), vials containing positive and negative control solutions for quality testing the device(s), a timer that may be used to determine when the assay of the invention is complete, a urine collection container (e.g., a urine sample container or other collection device), one or more transfer pipettes and/or a biohazard disposal container.

Although the foregoing invention has been described in some detail by way of illustration and example for clarity and understanding, it will be readily apparent to one of ordinary skill in the art in light of the teachings of this invention that certain changes and modifications may be made thereto without departing from the spirit and scope of the appended claims.

Experimental

As can be appreciated from the disclosure provided above, the present invention has a wide variety of applications. Accordingly, the following examples are offered for illustration purposes and are not intended to be construed as a limitation on the invention in any way. Those of skill in the art will readily recognize a variety of non-critical parameters that could be changed or modified to yield essentially similar results.

Example 1

To demonstrate the effectiveness of the methods and agents of the present invention in the reduction in false positive reactivity in an HIV-1 urine antibody test with the use of oxidative reagents.

This example demonstrates that the immunoassay devices of the present invention have a reduced rate of false positives over prior art methods.

Immunoassay devices of the invention was constructed from the following components:

A glass fiber sample zone pad, blocked and loaded with buffer by impregnating the pad with a solution containing 40% chicken serum (heat inactivated and filtered) in potassium phosphate buffer, 0.2% tectronic T-904. In examples wherein the oxidative agent was added to the analyzing device, the buffer also comprised 1 mM potassium stannate and 0.2% urea hydrogen peroxide. The pad was squeezed to remove excess liquid and allowed to dry-overnight at 30° C.

Analysis zone pads include HIV antigens coupled to a spun polyester membrane using a strepavidin/biotin linkage. Briefly, avidin was prepared at a 100 mg/ml solution. The HIV-1 and HIV-2 peptides were prepared each at 10 mg/ml. Avidin and the HIV peptides were mixed together at a ratio of 2.1 avidin binding site equivalents to 1 biotin moiety. The reaction was carried out at room temperature (25° C.) for five minutes. The solutions were brought to their final volumes using a DI water/5% isopropyl alcohol solution. These solutions were then striped to the membrane using a linear striper. The membrane was dried for four hours at 50° C. and blocked overnight in blocking solution (0.01 M potassium phosphate solution, with 0.25% BSA and 0.025% tween-20) overnight at 50° C.

The conjugation zone pads were prepared from spun-bonded polyester membranes by striping label moiety onto the pad using an aerosol tip. Prior to striping, the label moiety was stabilized using sucrose at 20% w/v and at 5% w/v trehalose (trehalose is a disaccharide used as a stabilizer and thickener). The pad was then dipped in a buffer containing polyvinlypyrollidone, chicken serum, bovine serum albumin, and carbonate buffer and dried at 50° C. using forced air for 50 minutes.

Control lines in the control zones of the devices were prepared by diluting an F(AB) fragment of a goat antibody specific to the Fc fragment of Human antibodies in water. The resulting solution was then sprayed onto nitrocellulose using an aerosol tip. The membrane was dried for four hours at 50° C. and blocked overnight in blocking solution (0.01M potassium phosphate solution, with 0.25% BSA and 0.025% tween20) overnight at 50° C.

The resulting membranes were then aligned in fluid communication relative to each other with the sample zone being upstream from the conjugation zone;

the conjugation zone upstream from the analysis zone; and the analysis zone upstream from the control zone.

The device was operated by adding four drops (50-100 μl) of urine (or, in certain tests, urine comprising the oxidative agent) to the sample zone. The result was be read from the device after 20 and 45 min at room temperature. A positive signal (e.g., colored line) that appeared at the control line meant that the test was functioning properly. In the rare event that there was no positive signal at the control line then the sample may not have contained antibody or the device was faulty and was discarded and the immunoassay was redone with a new device.

A positive signal in the analysis zone corresponding to the antigen was indicative of the presence of antibodies in the urine sample directed against the antigen. In the present analysis, this result indicated that the urine sample donor was infected with HIV. If a positive signal failed to appear in the analysis zone the result was indicative of an absence of antibodies in the urine sample directed against the antigen; i.e., that the donor of the urine sample was not infected with HIV.

The current example used urine specimens demonstrating false positive reactivity when tested with Cal 2003-108 HIV-1 rapid urine antibody test (Calypte Biomedical Corp., Pleasanton, Calif.). Oxidizing reagents were either added to the urine sample from stock solutions or the oxidative agents were added to the pads as described above.

In one set of tests, the dipstick type analyzing device was contacted to 250 μl of a urine specimen comprising a dilution of a stock solution of an oxidative reagent such that the sample zone of the lateral flow device was in contact with the urine specimen/oxidative, reagent solution long enough so that the sample zone was wetted. All stock solutions of the oxidative reagents were at 10% (unless noted differently below) and the resulting additions of 1, 5 and 10 μl to the 250 μl specimen, which resulted in final concentrations of 0.04%, 0.2% and 0.4%, respectively. At 20 and 45 min the test strips were interpreted for reactivity at the analysis and control zones. The presence of reactivity at the control zone is required for a valid test result. The absence of reactivity at the analysis zone indicates a negative result whereas the present of two bands at the control and analysis zones indicates a positive result. Controls were conducted where the oxidative reagents were not added to the urine specimens.

The oxidizing reagents used were selected from 10% hydrogen peroxide, 50% sucrose with 10 mg/ml glucose oxidase, 10% potassium perchlorate and 10% potassium permanganate, 10% potassium bromide, 10% potassium iodate, 10% potassium nitrate, 10% potassium nitrite, 10% urea hydrogen peroxide, 10% potassium superoxide 0.45% calcium bromate and 10% Thimerosol.

The oxidative reagents hydrogen peroxide, urea hydrogen peroxide and 0.2% urea hydrogen peroxide stabilized with 3% potassium stannate, 0.1% potassium iodate, 1% sucrose with 0.8 mg/ml glucose oxidase, 0.4%/potassium superoxide and 0.4% potassium permanganate, all at varying concentrations (as indicated above) were effective in reducing false positives while retaining true positive reactions

An additional set of experiments tested the effectiveness of the addition of the oxidative agent to the sample pad of the analyzing device (as described above). The oxidative reagents hydrogen peroxide, urea hydrogen peroxide and 0.2% urea hydrogen peroxide stabilized with 3% potassium stannate, 0.12% potassium periodate, 1% sucrose with 0.8 mg/ml glucose oxidase, 0.4% potassium superoxide and 0.4% potassium permanganate at varying concentrations (as indicated above) were effective in reducing false positives while retaining true positive reactions.

An additional set of experiments tested the effectiveness of the addition of the oxidative agent to the sample pad of the analyzing device (as described above). The oxidative reagents hydrogen peroxide, urea hydrogen peroxide and 0.2% urea hydrogen peroxide stabilized with 3% potassium stannate, 0.12% potassium periodate, 1% sucrose with 0.8 mg/ml glucose oxidase, and 0.4% potassium superoxide were effective in reducing false positives while retaining true positive reactions. See, Tables 1-3.

In should be evident from the forgoing that the present invention provides devices and methods for the detection of antibodies in fluids with the reduction of false positives and minimal or no reduction of true positives.

TABLE 1
Data Summary for Urine Samples with Oxidizing Agents

| | | Positive | | |
|---|---|---|---|---|
| Oxidizing | Percent | Control | Neg/False | HIV-1 |
| Agent | Concentration | Zone* | Positive** | Positive*** |
| Control | 0 | Black | False Positive | Positive |
| Hydrogen | 0.04 | Valid | Neg | Positive |
| Peroxide | 0.2 | Valid | Neg | Positive |
| | 0.4 | Valid | Neg | Positive |
| Urea Hydrogen | 0.1 | Valid | Neg | Positive |
| Peroxide | 0.4 | Valid | Neg | Positive |
| Sucrose + | 1.0 + 0.4 | Valid | Neg | NA |
| Glucose Ox. | 1.0 + 0.8 | Valid | Neg | NA |
| Thimerosol | 0.4 | Valid | Neg | Positive |
| Potassium | 0.4 | Valid | Neg | NA |
| Superoxide | | | | |
| Potassium | 0.1 | Valid | Neg | Positive |
| Iodate | 0.4 | Valid | Neg | Positive |
| Potassium | 0.4 | Valid | Neg | NA |
| Permanganate | | | | |
| Calcium | 0.045 | Valid | Neg | NA |
| Bromide | 0.09 | Valid | Neg | NA |
| | 0.18 | Valid | Neg | NA |

*Control band is valid if present in the control zone and pink to red in color.
**A negative result is the absence of a band at the test zone.
***Low titer plasma panel for HIV-1 spiked into urine.

TABLE 2
Data Summary for Oxidizing Agent in Sample Pad*

| | | Positive | | |
|---|---|---|---|---|
| | Percent | Control | Neg/False | HIV-1 |
| Oxidizing Agent | Concentration | Zone** | Positive*** | Positive**** |
| Urea Hydrogen | 0.2 | Valid | Neg | Positive |
| Peroxide + | 1 | | | |
| Potassium | | | | |
| Stannate | | | | |
| Potassium Iodate | 0.12 | Valid | Neg | Positive |

*Sample pads contain 0.2% Tween 80, 40% chicken serum, 0.25 M KCO3 pH 8.2, 0.05 M Potassium Phosphate in addition to the oxidizing agent formulation.
**Control band is valid if present in the control zone and pink to red in color.
***A negative result is the absence of a band at the test zone.
****Low titer plasma panel for HIV-1 spiked into urine.

TABLE 3
Data Summary for Subset of Oxidizing Agents in False Positive
Urine that Showed Results in the Absence of a Valid Control.

| | Percent | Positive | Neg/False |
|---|---|---|---|
| Oxidizing Agent | Concentration | Control Band* | Positive** |
| Potassium Nitrite | 0.1 | Valid | Neg |
| | 0.2 | Valid | Neg |
| | 0.4 | Valid | Neg |
| Potassium Bromide | 0.1 | Valid | Neg |
| | 0.4 | Valid | Neg |
| Potassium Choride | 0.4 | Valid | Neg |

*Control band is valid if present in the control zone and pink to red in color.
**A negative result is the absence of a band at the test zone.

What is claimed is:

1. A method for detecting the presence or absence of a target ligand in a sample, the method comprising
a. providing a labeled binding agent characterized by the ability to bind a target ligand;
b. contacting the labeled binding agent of step a) with a sample suspected of containing the target ligand under conditions suitable for binding the target ligand to the labeled binding agent, wherein contact of the labeled binding agent with the sample occurs in the presence of an oxidizing agent; and
c. assessing the binding of the target ligand to the labeled binding agent, thereby detecting the presence or absence of the target ligand in the sample.

2. The method of claim 1 wherein the sample is a biological sample.

3. The method of claim 2 wherein the biological sample comprises a biological fluid selected from the group consisting of urine, blood, and oral fluid.

4. The method of claim 1, wherein the target ligand is a protein.

5. The method of claim 4, wherein the protein is an antibody.

6. The method of claim 4, wherein the protein is a hormone.

7. The method of claim 1, wherein the target ligand is a non-protein.

8. The method of claim 7, wherein the non-protein is a lipid.

9. The method of claim 7, wherein the non-protein is a carbohydrate.

10. The method of claim 1 wherein the target ligand is an antibody to an HIV antigen.

11. The method of claim 1 wherein the labeled binding agent is attached to a solid support suitable for binding the target ligand to the labeled binding agent on said support.

12. The method of claim 1 wherein the labeled binding agent is movably supported on a surface.

13. The method of claim 12 wherein a chromatographic test strip comprises said surface.

14. The method of claim 12 wherein an immunochemical sampling device comprises said surface.

15. The method of claim 13 wherein a lateral flow device comprises the chromatographic strip.

16. The method of claim 13 wherein the sample is applied to the chromatographic test strip at a sample site and transported by sorption or capillary action along said strip prior to contact of the labeled binding reagent with the sample at a conjugation site.

17. The method of claim 1 wherein the oxidizing agent is selected from the group consisting of hydrogen peroxide, urea hydrogen peroxide, potassium chlorate, thimerosol, potassium iodate, potassium superoxide, potassium permanganate, sucrose containing glucose oxidase, calcium bromate, potassium chromate, potassium nitrate, potassium perchlorate and potassium permanganate.

18. The method of claim 17 wherein the oxidizing agent is hydrogen peroxide and the source of the hydrogen peroxide is urea hydrogen peroxide.

19. The method of claim 1 wherein contact of the labeled binding agent with the sample further occurs in the presence of a stabilizing agent.

20. The method of claim 19 wherein the stabilizing agent comprises potassium stannate.

21. The method of claim 1 wherein the labeled binding agent comprises a colloidal gold conjugate.

22. The method of claim 1 wherein the labeled binding agent comprises a Protein A conjugate.

23. The method of claim 1 wherein the oxidizing agent is solubilized from a solid dried on the test strip.

24. The method of claim 23 wherein the oxidizing agent is dried on the test strip at the conjugation site prior to applying said sample.

25. The method of claim 23 wherein the oxidizing agent is dried on the test strip at the sample site prior to applying said sample.

26. An immunochemical sampling device enabling detection of a target ligand in a biological sample, the device comprising a chromatographic test strip, the chromatographic test strip comprising:
a. a sample application zone;
b. a conjugate zone comprising a movably supported, labeled first binding agent that binds a target ligand of interest;
c. an analysis zone comprising a second binding agent immobilized therein which specifically binds the target ligand of interest; and
d. optionally, a control zone;
wherein, said sample application zone, conjugate zone, analysis zone, and control zone define a flow path for the sample and wherein, the chromatographic test strip comprises an oxidizing agent, or oxidizing agent source thereof, movably supported on the test strip so that contact of the labeled first binding agent with the sample occurs in the presence of the oxidizing agent.

27. The immunochemical sampling device of claim 26 wherein the biological sample comprises a biological fluid selected from the group consisting of urine, blood, and oral fluid.

28. The immunochemical sampling device of claim 26 wherein the target ligand is an antibody to an HIV antigen.

29. The immunochemical sampling device of claim 26 wherein a lateral flow device comprises the chromatographic strip.

30. The immunochemical sampling device of claim 26 wherein the oxidizing agent is selected from the group consisting of hydrogen peroxide, potassium chlorate, potassium bromate, potassium iodate, potassium periodate, potassium superoxide, potassium perm anganate, glucose oxidase, calcium bromate, potassium chromate, potassium nitrate, potassium perchlorate and potassium manganate.

31. The immunochemical sampling device of claim 26 wherein the oxidizing agent is hydrogen peroxide and the source of the hydrogen peroxide is urea hydrogen peroxide.

32. The immunochemical sampling device of claim 26 further wherein the oxidizing agent is dried on the chromatographic test strip.

33. The immunochemical sampling device of claim 30 wherein the oxidizing agent further comprises a stabilizing agent.

34. The immunochemical sampling device of claim 33 wherein the stabilizing agent comprises potassium stannate.

35. The immunochemical sampling device of claim 26 wherein the labeled first binding agent comprises a colloidal gold conjugate.

36. The immunochemical sampling device of claim 26 wherein the labeled first binding agent comprises a Protein A conjugate.

37. The immunochemical sampling device of claim 26 wherein the sample application zone comprises the oxidizing agent, or oxidizing agent source thereof, dried on the test strip.

38. The immunochemical sampling device of claim 26 wherein the conjugate zone comprises the oxidizing agent, or oxidizing agent source thereof, dried on the test strip.

39. The method of claim 26, wherein the target ligand is a protein.

40. The method of claim 39, wherein the protein is an antibody.

41. The method of claim 39, wherein the protein is a hormone.

42. The method of claim 26, wherein the target ligand is a non-protein.

43. The method of claim 42, wherein the non-protein is a lipid.

44. The method of claim 42, wherein the non-protein is a carbohydrate.

45. The immunochemical sampling device of claim 28 wherein the test zone comprises an HIV-1 gp41 synthetic peptide.

46. The immunochemical sampling device of claim 28 wherein the test zone comprises an HIV-1 gp41 recombinant protein.

47. The immunochemical sampling device of claim 28 wherein the test zone comprises an HIV-2 gp36 synthetic peptide.

48. The immunochemical sampling device of claim 28 wherein the test zone comprises an HIV-2 gp36 recombinant protein.

49. The immunochemical sampling device of claim 26 wherein the control zone comprises Protein A.

50. The immunochemical sampling device of claim 26 wherein the control zone comprises a goat anti-human IgG.
